(19) United States
(12) Patent Application Publication
(10) Pub. No.: US 2009/0053756 A1
(43) Pub. Date: Feb. 26, 2009

(54) METHOD FOR MONITORING AND PROMOTING THE NUTRITION AND WELL-BEING AS WELL AS THE PRODUCTIVITY OF ANIMALS

(75) Inventors: Markku Virkki, Espoo (FI); Jussi Vaahtovuo, Turku (FI); Mika Korkeamaki, Riihikoski (FI); Juhani Vuorenmaa, Tampere (FI); Eveliina Munukka, Turku (FI); Pirkko Hameenoja, Espoo (FI)

Correspondence Address: Studebaker & Brackett PC, 1890 Preston White Drive, Suite 105, Reston, VA, 20191, US

(73) Assignee: CYFLO OY, Turku, FI (FI)

(21) Appl. No.: 12/158,078

(22) PCT Filed: Dec. 21, 2006
(86) PCT No.: PCT/FI2006/000418

§ 371 Date: Sep. 11, 2008

(30) Foreign Application Priority Data
Dec. 22, 2005 (FI) FI20051319

(51) Int. Cl. C12Q 1/02; A23K 1/18

(52) U.S. Cl. 435/29; 426/2; 426/531

(57) ABSTRACT

The invention concerns a method for determining a characteristic describing the intestinal microbiota for an animal and/or a human being, in which method an animal and/or a human being is fed with a fodder/food; the number of two or more microorganisms in the intestine is determined; and the characteristic describing the intestinal microbiota is calculated as a ratio of the number of microorganisms. The invention also concerns methods for monitoring, development and promoting the intestinal microbiota, the well-being of an animal and/or a human-being, the intestinal health, the nutrition of an animal and/or a human being, the productivity and/or fodder utilization ratio of an animal, as well as the use of the characteristic and the use of the method for developing a fodder, a nutriment, a fodder and nutritional additive, a preparation and a pharmaceutical supporting the well-being, and a nutritional program.

### FIELD OF THE INVENTION

The invention relates to a method defined in the preamble of claim 1 for determining a characteristic describing the intestinal microbiota. Further, the invention relates to a method for monitoring the intestinal microbiota, the well-being of an animal and/or a human being, the intestinal well-being, intestinal health, the nutrition of an animal and/or a human being, the productivity and/or the fodder utilization ratio of animals; a method for promoting the nutrition, the intestinal microbiota, the intestinal health and/or the well-being of an animal and/or a human being and/or for enhancing the productivity and/or the fodder utilization ratio of an animal; the use of the characteristic; the use of the method for developing a fodder, a nutriment, a nutritional additive, a fodder additive, a preparation and a pharmaceutical supporting the well-being; and a nutritional program.

### BACKGROUND OF THE INVENTION

The intestinal microbiota and its composition have an essential effect on the health and well-being of animals and human beings, as well as on the productivity of animals. Symptoms of the impairment of intestinal health include diarrhea and other intestine-related health problems, which for animals can lead e.g. to the death of the animal.

The intestinal microbiota and its composition can also be in the background of many diseases whose etiology is unknown or whose are partly unknown. For human beings, examples of these diseases include allergy, diabetes, inflammatory intestinal diseases and intestinal cancers.

The intestinal microbiota is a highly complicated ecosystem. The microbiota consists of hundreds of bacterial species, and their bacterial density is uniquely high, even 10¹¹/g. The intestine has been estimated to contain an abundance of bacterial species unknown this far. Known bacteria are divided into genera or groups and further into bacterial species based on their 16S rDNA. The 16S rDNA is a conservative gene sequence coding the structures of ribosomes, which sequence is similar for closely related bacteria. The most common bacterial genera and groups to be found in intestinal samples include several different Bacteroides genera, Clostridium genera, the Bifidobacterium genus, the Lactobacillus genus, the enteric group, etc. Bacterial genera and groups are further divided into bacterial species, such as the Bacteroides thetaiotaomicron and Bifodobacterium animalis.

A usual way to examine the intestinal microbiota is to analyze and list individual bacteria to be found in the intestine, usually included in the harmful bacterial species.

A problem with the prior-art way is that the analysis of bacteria included in individual bacterial species does not take into account the total population of bacteria in the intestine. Individual bacterial species seldom are in the background of changes of intestinal health alone, and the analysis of bacteria included in individual bacterial species does not give a general picture of the intestinal microbiota.

### OBJECTIVE OF THE INVENTION

It is an objective of the present invention to eliminate the problems referred to above.

One specific objective of the invention is to disclose a new type of method for determining a characteristic for an animal and/or a human being that would comprehensively describe the intestinal microbiota, its balance, composition and/or changes.

It is a further objective of the invention to disclose a method for monitoring the intestinal microbiota, the well-being of an animal and/or a human being, the intestinal well-being, the intestinal health, the nutrition of an animal and/or a human being, the productivity and/or the fodder utilization ratio of an animal, which method enables one to clearly detect the changes in the microbiota, the well-being, intestinal well-being, intestinal health, nutrition, productivity and fodder utilization ratio.

The objective of the invention is to also disclose a method for promoting the nutrition, the intestinal microbiota, the intestinal well-being and well being of an animal and/or a human being and/or for enhancing the productivity and the fodder utilization ratio of an animal, which method enables one to affect the intestinal microbiota to support and promote the well-being and health of an animal and/or a human being and/or to support and enhance the productivity of animals.

It is a further objective of the invention to disclose the use of a characteristic of the invention based on the number of microorganisms in the intestine for optimally monitoring, developing and/or promoting the intestinal microbiota, intestinal health, the well-being of an animal and/or a human being, the nutrition of an animal and/or a human being, the productivity and/or the fodder utilization ratio of an animal.

It is still another objective of the invention to disclose the use of a method of the invention for developing a fodder, a nutriment, a fodder additive, a nutritional additive promoting the well-being, intestinal health and/or the productivity, a preparation and a pharmaceutical promoting the well-being, as well as a nutritional program.

### SUMMARY OF THE INVENTION

The method of the invention for determining a characteristic describing the intestinal microbiota is characterized by what is presented in claim 1.

The method of the invention for monitoring the intestinal microbiota, the well-being of an animal and/or a human being, the intestinal health, the nutrition of an animal and/or a human being, the productivity and/or the fodder utilization ratio of an animal is characterized by what is presented in claim 2.

The method of the invention for promoting the nutrition, the intestinal microbiota, intestinal health and/or the well-being of an animal and/or a human being and/or enhancing the productivity and/or the fodder utilization ratio of an animal is characterized by what is presented in claim 3.

The use of a characteristic of the invention based on the number of microorganisms in the intestine for monitoring, developing and/or promoting the intestinal microbiota, the intestinal health, the well-being of an animal and/or a human being, the nutrition of an animal and/or a human being, the productivity and/or the fodder utilization ratio of an animal is characterized by what is presented in claim 12.

The use of a method of the invention for developing a fodder, a nutriment, a fodder additive, a nutritional additive, a preparation and a pharmaceutical promoting the well-being, as well as the nutritional program are characterized by what is presented in claims 13 to 21.

The invention is based on research work conducted, where one examined the effect of feeding on the intestinal microbiota of pigs and where one surprisingly found that from the number of bacteria in the intestine it is possible to calculate a characteristic describing the intestinal microbiota, the characteristic positively correlating with the well-being and growth of the animal and negatively with the fodder utilization ratio.

In the method of the invention, in determining the characteristic describing the intestinal microbiota, an animal/and or a human being is fed with a fodder/fod and thereafter the number of two or more microorganisms in the intestine is determined. Further, in the method, the characteristic describing the intestinal microbiota is calculated as a ratio of the number of microorganisms.

The characteristic calculated by the method of the invention can be used to monitor, develop and/or promote the intestinal microbiota, the intestinal health, the well-being of an animal and/or a human being, the nutrition of an animal or a human being, the productivity and/or the fodder utilization ratio of an animal. When monitoring the intestinal microbiota, one monitors the balance, composition and/or the changes in the microbiota. The nutrition includes and is affected by the feeding, fodders, fodder substances, fodder additives, feed programs, nourishment, nutritional supplements, preparations supporting the well-being such as intestinal improvers and preparations modifying the intestinal microbiota, pharmaceuticals and/or the like. Productivity is used to mean an increase in the yield such as in the weight/meat amount, milk amount, egg amount, fur and/or similar yield. The fodder utilization ratio is used to mean the proportion of the amount of fodder consumed by an animal to the yield.

In the method of the invention for monitoring the intestinal microbiota, the well-being of an animal and a human being, the intestinal well-being, the intestinal health, the nutrition of an animal and a human being, the productivity and/or the fodder utilization ratio of an animal, an animal and/or a human being is fed with a fodder/food and thereafter the number of two or more microorganisms in the intestine is determined and the characteristic is calculated as a ratio of the number of microorganisms. The intestinal microbiota, the well-being of an animal and a human being, the intestinal well-being, the intestinal health, the nutrition of an animal and a human being, the productivity and/or the fodder utilization ratio of an animal are monitored by comparing the computed characteristic to known characteristics compiled for different fodders/foods, for which characteristics, the nutrition, well-being, productivity and/or fodder utilization ratio to be achieved by them and/or correlating with them have been determined, as well as to the productivity and/or the fodder utilization ratio.

In the method of the invention for promoting the nutrition, the intestinal microbiota, the intestinal well-being and/or the well-being of an animal and/or a human being and/or for enhancing the productivity and/or the fodder utilization ratio of an animal, an animal and/or a human being is fed with a fodder/food and thereafter the number of two or more microorganisms in the intestine is determined. Further, in the methods, from the number of microorganisms, the characteristic describing the intestinal microbiota is calculated as a ratio of the number of microorganisms, and the characteristic is compared to the characteristics correlating with the nutrition, the intestinal microbiota, the intestinal well-being and/or the well-being, productivity and/or fodder utilization ratio. Based on the comparison, a fodder/food maintaining and/or improving the nutrition, well-being, productivity and/or fodder utilization ratio is selected as the fodder.

The developing, maintaining and improving of the nutrition, intestinal microbiota, intestinal well-being, well-being, productivity and/or fodder utilization ratio, according to the invention, also means the developing, maintaining and improving of the corresponding correlating characteristics.

Based on the correlations, productivity and/or fodder utilization ratio, feed that supports a beneficial microbiota is selected as the feed. Because the composition of the microbiota has an essential effect on the intestinal health and the intestinal health is a prerequisite for the health and well-being of an animal and a human being as well as for the productivity of an animal, the control of the microbiota can be used to favorably affect the intestinal health and/or the well-being, productivity and/or the fodder utilization ratio parameters.

In one embodiment of the invention, the characteristic is compared to the characteristics describing the intestinal microbiota and correlating with the well-being, and as the feed of an animal and/or a human being, a fodder/food maintaining and/or improving the well-being is selected, and/or the feeding is complemented with a fodder substance, a fodder additive, a nutritional supplement maintaining and/or improving the well-being and/or with a preparation supporting the well-being.

In one embodiment of the invention, the characteristic is compared to the productivity and/or the fodder utilization ratio, and as the animal feed, a fodder/food maintaining and/or improving the productivity is selected, and/or the feeding is complemented with a fodder substance and/or a fodder additive maintaining and/or improving the productivity.

The characteristic is calculated for each fodder/food specifically.

The characteristic describing the microbiota and the well-being, productivity and/or fodder utilization ratio are determined separately for different animals, groups of animals, fodders and/or feeds.

The number of microorganisms in the intestine is determined from a fecal sample an/or a corresponding sample describing the composition of microorganisms in the intestine.

In the method, the characteristic describing the intestinal microbiota is calculated as a ratio of the number of two or more microorganisms using conventional arithmetic operations such as addition, subtraction, division and/or multiplication.

The characteristic describing the intestinal microbiota can also be calculated as a ratio of the number of microorganisms or as a ratio of the amounts of some other physiological variable, including the dry matter content of the gut content, the composition of the gut content, some cell types of the organism, antibodies and/or transmitters.

Characteristic of a microbiota is that a change in the number and/or property of one microorganism is reflected in another microorganism/other microorganisms of the microbiota as well as in the environment such as the host animal. In stabile conditions, between the microorganisms of a microbiota there can be a balance prevailing, where the absolute and/or relative numbers of microorganisms remain substantially the same. In the methods of the invention, the characteristic describing the microbiota reflects the composition of the entire microbiota, and a change in the characteristic reflects a change in the balance/status of the entire microbiota and/or microbiota.

As the number of microorganisms, the absolute and/or relative number of microorganisms is used. The relative number of microorganisms can be calculated e.g. by comparing the number of microorganisms to the total number of microorganisms or to the total number in the dry matter.

In the method it is possible to use the number of one or more microorganisms beneficial and/or neutral to the intestine, i.e. ones that do not have a harmful effect on the host, and/or the number of one or more microorganisms harmful to the intestine. A beneficial, neutral or harmful microorganism is used to mean a microorganism that has been stated or is assumed to be beneficial, neutral or harmful to the intestine. The effect/effects of microorganisms can be direct or indirect.

A microorganism can be any microbe included in the mirobiota such as a bacterium, a protozoan, a fungus and/or a virus.

In one embodiment of the invention, the microorganism is a bacterium included in the bacterial genus, bacterial species, bacterial strain and/or a bacterial group. The bacterium can be e.g. a bacterium of the Bifidobacterium and/or the Lactobacillus genus, the Bacteroides-Porphyromonas-Prevotella group, the Clostridium leptum group, the Faeca-libacterium prausnitzii group, the enteric group and/or a bacterium of any other bacterial genus, bacterial species, bacterial strain and/or bacterial group included in the intestinal microbiota. From different bacterial genera, bacterial species and/or bacterial groups it is possible to select groups that are suited for the calculation of the characteristic.

In one embodiment of the invention, the characteristic is calculated by calculating the sum of the number of bacteria in at least one bacterial species, genus and/or group and by dividing the sum by the sum of the number of bacteria in at least one bacterial species, genus and/or group.

In one embodiment of the invention, the characteristic is calculated by summing up the sum of the number of bacteria of two bacterial species, genera and/or groups that are not supposed to have a harmful effect on the host and by dividing the sum by the sum of the number of bacteria of two bacterial species, genera and/or groups that are supposed to contain pathogenic bacteria or ones causing looseness of the gut content.

In one embodiment of the invention, the characteristic is calculated from the numbers of bacteria of the selected bacterial genera and groups as follows:

Characteristic=(Bif+Fprau)/(Bacto+Enter) (1)

Where Bif is the number of bacteria included in the Bifidobacterium genus;

Fprau is the number of bacteria included in the Faecalibacterium prausnitzii group;

Bacto is the number of bacteria included in the Bacteroides-Porphyromonas-Prevotella group; and

Enter is the number of bacteria included in the enteric group.

In the method, the number of microorganisms is counted by cytometry, a cultivation method, a microscopy and/or by any other corresponding method suited for counting the number of microorganisms.

In one preferred embodiment of the method, the number of microorganisms is counted by flow cytometry, which enables very accurate determination of the number of pieces of microorganisms. Determination based on flow cytometry is described e.g. in international patent application PCT/FI2003/000596.

The correlations between the characteristic of the microbiota and the well-being, productivity and/or fodder utilization ratio parameters are utilized, according to the invention, when planning and developing fodders, nutrients, nutritional and/or fodder additives achieving a beneficial microbiota in terms of the well-being, productivity and/or the fodder utilization ratio, as well as when planning and developing preparations, pharmaceuticals and/or nutritional programs supporting the well-being.

In one embodiment of the invention, the preparation supporting the well-being is an intestinal improver supporting the intestinal well-being.

In one embodiment of the invention, the preparation supporting the well-being is a preparation modifying the intestinal microbiota.

The nutritional and fodder additives developed, the preparations and pharmaceuticals supporting the well-being can be based e.g. on living and/or dead yeasts, hydrolyzed yeasts, various sugar structures, mixtures thereof, sugar alcohols as well as on other preparations containing microorganisms and/or prebiotics and/or on substances possessing direct and/or indirect microbial and/or microbiotic effects or similar substances.

The methods of the invention are suited for monitoring and developing the nutrition of various animals, including production animals such as pigs, specifically piglets, sheep, poultry such as hens, broilers and turkeys, ruminants and fishes, fur animals and hobby animals and pets such as cats, dogs and horses, as well as for monitoring and promoting the productivity of production animals.

With the method of the invention, the intestinal microbiota can be described comprehensively, pithily and intelligibly. Specifically by using flow cytometry, the number of pieces of microorganisms, such as representatives of bacterial genera and groups, and the microbiota of the intestine can be determined very accurately.

The characteristic calculated by the method describes the entire intestinal microbiota accurately because changes in the number of all intestinal microorganisms, also uncounted and unknown ones, are reflected in the number of the selected microorganisms such as bacteria.

Planning of nutrition that includes planning of feeding, fodders, nutritional and/or fodder additives, preparations, pharmaceuticals and nutritional programs supporting the well-being can be based on pursuit for the control of the microbiota, and thus it is possible to beneficially affect the intestinal health and well-being, the productivity and/or fodder utilization ratio. With the methods of the invention it is possible to optimize the feeding and nourishment of an animal and/or a human being so that it best supports and promotes the well-being, intestinal health and productivity of an animal. The optimization of the feeding also prevents from suffering from intestinal diseases and other diseases.

The feeding and fodder developed with the method of the invention promote the intestinal health and the microbial balance. In this manner, the growth of the animal, the fodder utilization and the overall efficiency of the production are increased. Further, environmental emissions of the production are decreased because the animal is able to take advantage of the fodder more efficiently. Moreover, the use of a fodder developed in accordance with the invention in the feeding of animals gives a possibility to stop using fodder antibiotics.

Further, the methods of the invention provide a parallel or an alternative method for the conventional ways of monitoring and developing the nutrition that are based on nutritional values, chemical compositions and energy value systems, as well as for describing the procedures.

### LIST OF FIGURES

FIG. 1 illustrates the effect of feeding on the growth of piglets and on the characteristic describing the intestinal microbiota; and

FIG. 2 illustrates the effect of feeding on the fodder utilization ratio and on the characteristic describing the intestinal microbiota.

### DETAILED DESCRIPTION OF THE INVENTION

### Example 1

The test studied the effect of feeding on the intestinal microbiota and growth of piglets as well as on the fodder utilization ratio.

Test Arrangements and Feeding

Seven groups of piglets participated in the test, each of them having 18 to 24 piglets. Each group of piglets was fed with a different fodder. There were no substantial differences in the nutritional values and chemical compositions of the test fodders (1-7). Except for a group of piglets (Test group 1) with an unbalanced intestinal microbiota, the feeding test was implemented in the same test farm. Groups of piglets 2-7 consisted of basically healthy animals.

The fodders differed from one another in terms of the fodder substances or fodder additives contained therein as well as in terms of the processing parameters of the fodder preparation, these including humidity, granular hardness, granular size, granular shape, preparation temperature, pressure, mixing, and preparation equipment. Common to all of the fodders were specific preparations promoting the intestinal health; the content, composition and processing parameters of which preparations had been modified. Table 1 represents the variables compared in the test arrangement.

TABLE 1

| Group | Fodder variables to be |
|---|---|
| Number of piglets | compared |
| 1 | Source of protein L |
| (18) | Acid mixture |
| | Intestinal improver P |
| | Processing Y, -, - |
| | Dosing C |
| 2 | Source of protein S |
| (24) | Acid mixture F |
| | Intestinal improver P |
| | Processing Y, E, K |
| | Dosing C |
| 3 | Source of protein L |
| (24) | Acid mixture M |
| | Intestinal improver P |
| | Processing X, D, T |
| | Dosing C |
| 4 | Source of protein L |
| (24) | Acid mixture F |
| | Intestinal improver P |
| | Processing X, D, T |
| | Dosing C |
| 5 | Source of protein L |
| (24) | Acid mixture M |
| | Intestinal improver P |
| | Processing X, E, T |
| | Dosing C |
| 6 | Source of protein L |
| (24) | Acid mixture M |
| | Intestinal improver P |
| | Processing Y, D, T |
| | Dosing B |
| 7 | Source of protein L |
| (24) | Acid mixture M |
| | Intestinal improver P |
| | Processing X, D, T |
| | Dosing B |

Determination

The bacterial calculation of the faecial samples of piglets (7 weeks) was implemented by flow cytometry using the method described in international patent application PCT/FI2003/00596.

The bacterial cells were separated from the non-bacterial material of the sample by dissolving the sample in a phosphate-buffered saline solution (PBS) for 2 hours at +4 degrees. After the dissolution, the sample was lightly centrifuged. The separated bacteria were fixed in a 4% paraformaldehyde solution over night at +4 degrees. After the fixation, the bacteria were washed three times with PBS and by centrifuging (13,000 rpm, 3 min.). The fixed and washed bacteria were transferred to a 50% ethanol-PBS.

Bacterium genus/bacterium group-specific identification of bacteria in the samples is based on 16S rRNA hybridization which used e.g. oligonucleotide probes labeled with fluorescent agents that identify bacteria of the Bacteroides-porphyromonas-Prevotella group, bacteria of the Bifidobacterium genus, bacteria of the so-called enteric group and bacteria of the Faecalibacterium prausnitzii group. After the hybridization, the bacteria were stained with a fluorescent agent binding to nucleic acids and analyzed with cytometry. From the samples, the total amounts of bacteria and the amounts and relative proportions of the representatives of different bacterial genera/groups were counted, and from these a characteristic was calculated using formula (I).

The growth of the piglets was determined by weighing and the fodder utilization ratio was determined by weighing and by consumption of fodder.

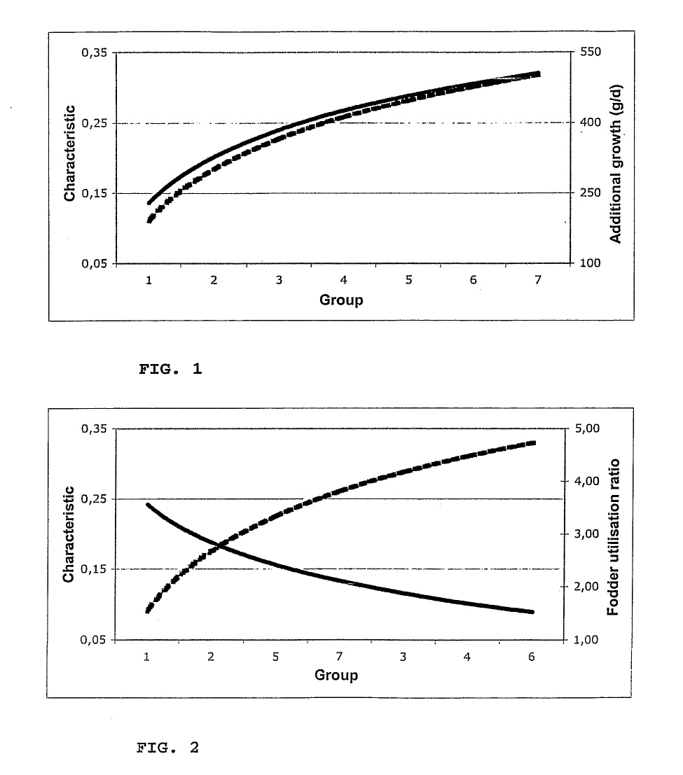

Table 2 shows the results of the test in numeric form and FIGS. 1 and 2 represent the results of the test in graphic form. In the figures, the trend line of the values of the characteristic is shown with a broken line. Based on variance analysis, the characteristics calculated for the test groups statistically significantly differed from one another (p<0.001). The correlation coefficient for the characteristics and additional growths of the test groups was 0.93; the correlation coefficient for the characteristics and fodder utilization ratios was −0.95; and the correlation coefficient for the additional growth and fodder utilization ratio was 0.97. The correlation coefficients were statistically significant.

TABLE 2

| | Additional | Fodder utilization | |
|---|---|---|---|
| Test | growth | ratio (g/d)/additional | |
| group | (g/d) | growth (g/d) | Characteristic |
| 1 | 200 | 4.00 | 0.07 |
| 2 | 343 | 2.51 | 0.19 |
| 3 | 418 | 1.89 | 0.27 |
| 4 | 454 | 1.86 | 0.33 |
| 5 | 456 | 1.94 | 0.25 |
| 6 | 461 | 1.85 | 0.31 |
| 7 | 484 | 1.93 | 0.26 |

Table 2 and the figures show that the combination “L, M, P, X, D, T, B” (Test group 7) of variables shown in Table 1 achieved the fourth best fodder utilization ratio and the fourth biggest value of the characteristic.

The combination “L, M, P, Y, D, T, B” of variables shown in Table 1 (Test group 6) achieved the best fodder utilization ratio and the second best value of the characteristic.

Based on these results, it is possible to plan e.g. a feed that can be used to support the composition of the intestinal microbiota of the animals in Group 7 towards a bigger value of the characteristic and a better fodder utilization ratio. This can be implemented e.g. by replacing processing parameter X of the fodder with processing parameter Y.

When performing a corresponding test for different animals/fodders it is possible by comparing various variables in the same manner to optimize for each animal group a feed favoring a bigger value of the characteristic, a better growth and/or a better fodder utilization ratio.

The invention is not limited merely to the examples referred to above, but many modifications are possible within the scope of the inventive idea defined by the claims.

1. A method for determining for an animal and/or a human being a characteristic describing the intestinal microbiota, in which method
an animal/a human being is fed with a fodder/food
the number of microorganisms in the intestine is determined;
wherein the characteristic describing the intestinal microbiota is calculated as a ratio of the number of microorganisms according to the following equation:
Characteristic=(Bif+Fprau)/(Bacto+Enter), where
Bif is the number of bacteria included in the Bifidobacterium genus;
Fprau is the number of bacteria included in the Faecalibacterium prausnitzii group;
Bacto is the number of bacteria included in the Bacteroides Porphyromonas-Prevotella group; and
Enter is the number of bacteria included in the enteric group.

2. A method for monitoring the intestinal microbiota, the nutrition of an animal and/or a human being, the productivity and/or the fodder utilization ratio of an animal, the method comprising the steps of:
feeding the animal/human being with a fodder/food;
determining the number of microorganisms ir. the intestine;
wherein the characteristic describing the intestinal microbiota is calculated as a ratio of the number of microorganisms according to the following equation:
Characteristic=(Bif+Fprau)/(Bacto+Enter), where
Bif is the number of bacteria included in the Bifidobacterium genus;
Fprau is the number of bacteria included in the Bacteroides Porphyromonas-Prevotella group; and
Enter is the number of bacteria included in the enteric group;
the characteristic is compared to the characteristics correlating with the nutrition, the intestinal microbiota, productivity and/or fodder utilization ratio.

3. A method for promoting the nutrition, the intestinal microbiota of an animal and/or a human being and/or for enhancing the productivity and/or fodder utilization ratio of an animal, the method comprising the steps of:
feeding the animal/human being with a fodder/food;
determining the number if microorganisms in the intestine;
wherein the characteristic describing the gut microbiota is calculated as a ratio of the number of microorganisms according to the following equation:
Characteristic=(Bif=Fprau)/(Bacto+Enter), where
Bif is the number of bacteria included in the Bifidobacterium genus;
Fprau is the number of bacteria included in the Faecalibacterium prausnitzii group;
Bacto is the number of bacteria included in the Bacteroides Porphyromonas-Prevotella group; and
Enter is the number of bacteria included in the enteric group;
the characteristic is compared to the characteristics correlating with the nutrition, intestinal microbiota, productivity and/or fodder utilization ratio;
the animal/human being is fed with a fodder/food that maintains and/or improves the nutrition, intestinal microbiota, productivity and/or fodder utilization ratio.

4. The method as defined in claim 3, wherein the nutrition is developed by comparing the characteristic to the characteristics correlating with the nutrition and/or intestinal microbiota, and the animal/human being is fed with a fodder/food that maintains and/or improves the nutrition and/or intestinal microbiota, and/or the feeding is complemented with a fodder, a fodder additive, a nutritional supplement that maintains and/or improves the nutrition and/or intestinal microbiota and/or with a preparation that supports the nutrition and/or intestinal microbiota.

5. The method as defined in claim 3, wherein the productivity of the animal is promoted by comparing the characteristic to the productivity and/or the fodder utilization ratio, and the animal is fed with a fodder/food that maintains and/or improves the productivity, and/or the feeding is complemented with a fodder substance and/or a fodder additive that maintains and/or improves the productivity.

6. The method as defined in claim 1, wherein the number of microorganisms is counted with flow cytometry.

7. A characteristic determined using a method in accordance with claim 1 to be used to monitor, develop and/or promote the intestinal microbiota, intestinal health, the well-being of an animal and/or a human being, the productivity and/or fodder utilization ratio of an animal.

8. The use of a method in accordance with claim 3, for developing a fodder.

9. The use of a method in accordance with claim 3, for development a nutriment.

10. The use of a method in accordance with claim 3, for developing a nutritional additive.

11. The use of a method in accordance claim 3, for developing a fodder additive.

12. The use of a method in accordance with claim 3, for developing a preparation that supports the well-being.

13. The use as defined in claim 12, wherein the preparation is an intestinal improver supporting the intestinal health.

14. The use as defined in claim 12, wherein the preparation is a preparation that modifies the intestinal microbiota.

15. The use of a method in accordance with claim 3, for developing a pharmaceutical.

16. A nutritional program where for the feeding of an animal and/or a human being, a nutriment, a fodder, a nutritional and/or fodder additive and/or a preparation supporting the nutrition and/or intestinal microbiota developed by a method in accordance with claim 3, are used.
